(19) United States
(12) Patent Application Publication
(10) Pub. No.: US 2003/0172933 A1
(43) Pub. Date: Sep. 18, 2003

(54) APPARATUS FOR SHAPING A LARYNGEAL MASK

(76) Inventor: GARRY HAMILTON NIMMO, BANBURY (GB)

Correspondence Address: PAUL E MILLIKEN, 9061 WALL STREET NW, MASSILLON, OH, 44646-1676, US

(21) Appl. No.: 09/463,866

(22) PCT Filed: Jul. 31, 1998
(86) PCT No.: PCT/GB98/02158

(30) Foreign Application Priority Data
Aug. 2, 1997 (GB) 9716287.9

(51) Int. Cl. A61M 16/00

(52) U.S. Cl. 128/207.14; 128/200.26; 128/207.15

(57) ABSTRACT

Apparatus for shaping the deflated profile of a laryngeal mask (10) and which comprises a body (20) having a cavity (21) therein, the mouth (22) of the cavity (21) being shaped to accommodate the elliptical outline of an inflated mask in an inverted condition, and having sufficient depth (d) to accommodate the inverted mask and air tube. The sides (26-31) of the cavity preferably incline inwardly. If an inverted mask is deflated and simultaneously pushed downwards into the cavity an optimized shape for the deflated mask is achieved.

### FIELD

This invention relates to apparatus for shaping laryngeal masks.

### BACKGROUND

A laryngeal mask is an artificial airway that facilitates lung ventilation in an anesthetized patient. A known laryngeal mask is described in British Patent 211 324B and comprises a curved flexible tube with a mask portion carried at one end of the tube. The mask portion has an elliptical base with an opening connected to the tube and which is surrounded by a peripheral inflatable annular collar or cuff. The device is capable of conforming to and fitting readily within the space behind the larynx and the tube opening through the base provides an airway.

Once the peripheral collar has been inflated the tube establishes an exclusive passageway to the patient's trachea.

Such devices have been successful in use. Insertion of the device has been found to be relatively easy, but not without problems. In use the mask portion is passed, in a deflated condition, through the patient's mouth into the pharynx, and engages at the upper oesophageal sphincter. Subsequent inflation then causes the collar to establish a desired seal to the laryngeal inlet.

A problem arises from the fact that an unskilled or careless user of the laryngeal mask may not be able to achieve a correctly collapsed shape of the deflated collar making insertion of the mask more difficult.

In order to overcome this problem it has been proposed in GB-A-2285 765 to provide a tool which forms the collar of the mask into a predetermined deflated configuration. The deflated configuration shown in GB-A-2285 765 is one in which the collar is a smooth continuous upwardly flared configuration. However this is not a perfect shape that fits naturally to the anatomical pathways in that the distal end of the cuff projects out of the smooth curve or the tube and base.

The present invention provides an apparatus for shaping a laryngeal mask to a shape more suited for insertion into the patient's larynx. The term “patient” may also include animals, where laryngeal masks are used in veterinary procedures.

### STATEMENTS OF INVENTION

According to the invention there is provided apparatus for shaping the deflated profile of a laryngeal mask and which comprises a body having a cavity therein, the mouth of the cavity being shaped to accommodate the elliptical outline of an inflated mask in an inverted condition, and the cavity having sufficient depth to accommodate the inverted mask and air tube. By “inverted” is meant a laryngeal mask with the opening in the base directed upwards with the tube located below the base.

Preferably the sidewalls of the cavity are inclined inwardly of the cavity. This gives a more consistent deflated mask shape than, say, if the walls were vertical. It also allows for a single apparatus to be utilised for more than one size of laryngeal mask.

Preferably the body of the apparatus is made from a block of resilient material e.g. an elastomeric material such as silicone rubber, a semi-rigid foam such as polyurethane or polyethylene foam. Alternatively the apparatus may be moulded, preferably by injection moulding or vacuum forming from a glass reinforced polyester resin (GRP) or may be moulded from a thermoplastic material such as high or low density polyethylene, polypropylene, or a mixture thereof, from polycarbonate, or ABS (acrylontrile butadiene styrene).

Preferably the upper sidewalls adjacent the mouth of the cavity are chamfered, the chamfered portion may form a continuous edge margin around the periphery of the cavity and is inclined inwardly at an angle of between 20-45 degrees, preferably about 30 degrees. In use the chamfered edges support an inflated cuff of a larger size mask, and the mask base fits within the mouth of the cavity.

The preferred shape of cavity is substantially hexagonal in plan view, preferably an irregular hexagon, with more preferably one end of the cavity connected to a slot open to the periphery of the body and which accommodates the tube. The base of the slot may be inclined downwardly to accommodate the tube, especially if the apparatus is formed from a more rigid material such as GRP, or polycarbonate.

The inwardly inclined sidewalls of the cavity help shape a deflating cuff as the mask is pressed lightly into the cavity and the corners of the hexagon also provide some assistance in the deflation and shaping process.

### DESCRIPTION OF DRAWINGS

The invention will be described by way of example and with reference to the accompanying drawings in which:

FIG. 1 is a side elevation of a known laryngeal mask in an inverted condition;

FIG. 2 is an isometric view of a prior art deflated condition for a laryngeal mask;

FIG. 3 is an isometric view of laryngeal mask deflated in apparatus according to the present invention,

FIG. 4 is a plan view of apparatus according to the present invention,

FIG. 5 is a section on the line V-V in FIG. 4, and

FIG. 6 is a section on the line VI-VI in FIG. 4.

### DETAILED DESCRIPTION OF THE INVENTION

With reference to FIG. 1 there is shown a prior art laryngeal mask 10 in an inverted condition, comprising a flexible airway tube 11 and a mask 12. Both the mask 12 and tube 11 are formed from a silicone rubber material. The mask 12 includes an inflatable cuff or collar 13 around its periphery. The tube 11 is connected to a hollow boss 14 in the back of the mask base 15 and opens into the face of the mask 12.

The inflatable cuff 13 is connected by a second smaller tube 16 to a pump device 17 for inflation and deflation of the cuff 13. A suitable pump device would be a syringe.

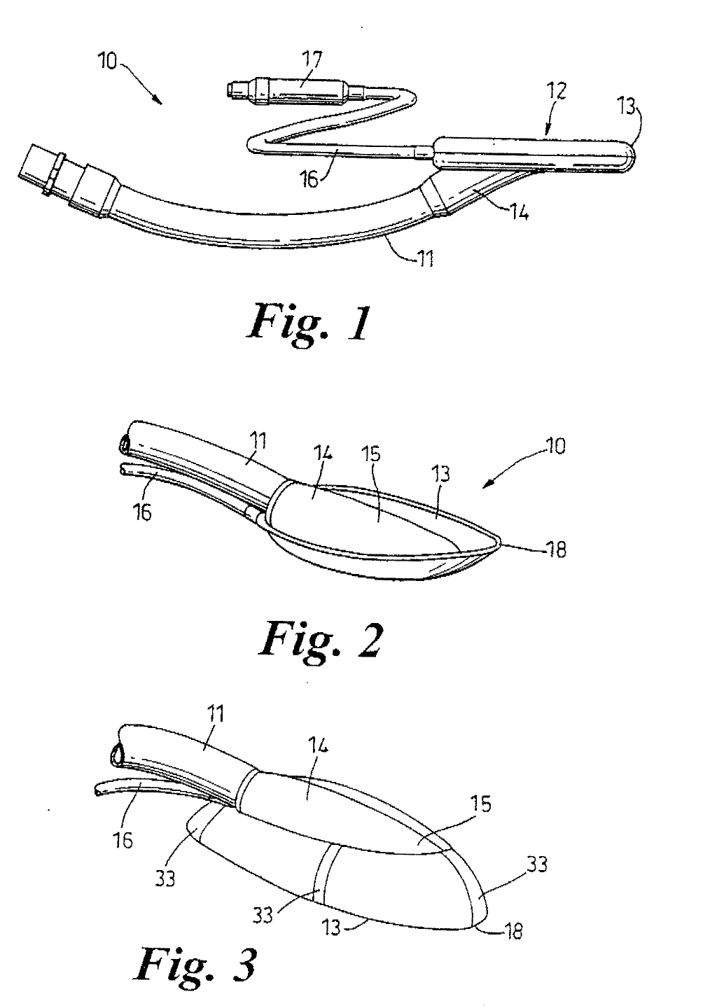

With reference to FIG. 2 there is shown a laryngeal mask 10 in which the cuff 13 has been deflated in a known manner. It can be seen that the cuff is flared (upwardly as shown) in the direction of the base 15 so that the tube 11, boss 14 and cuff 13, when deflated, do not form a smooth curve since the distal end 18 of the cuff 13 projects upwardly (as shown) of the curve.

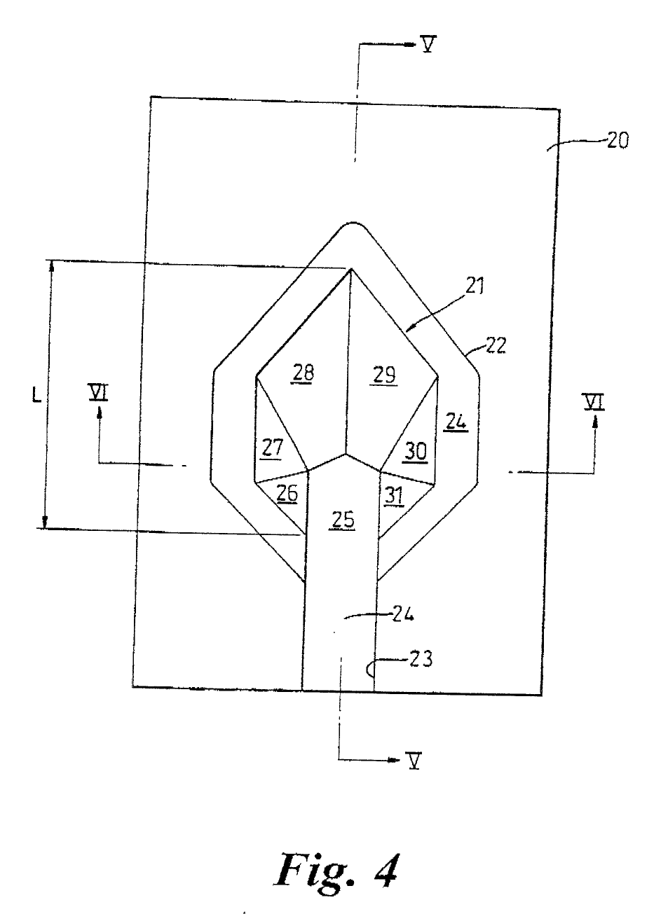

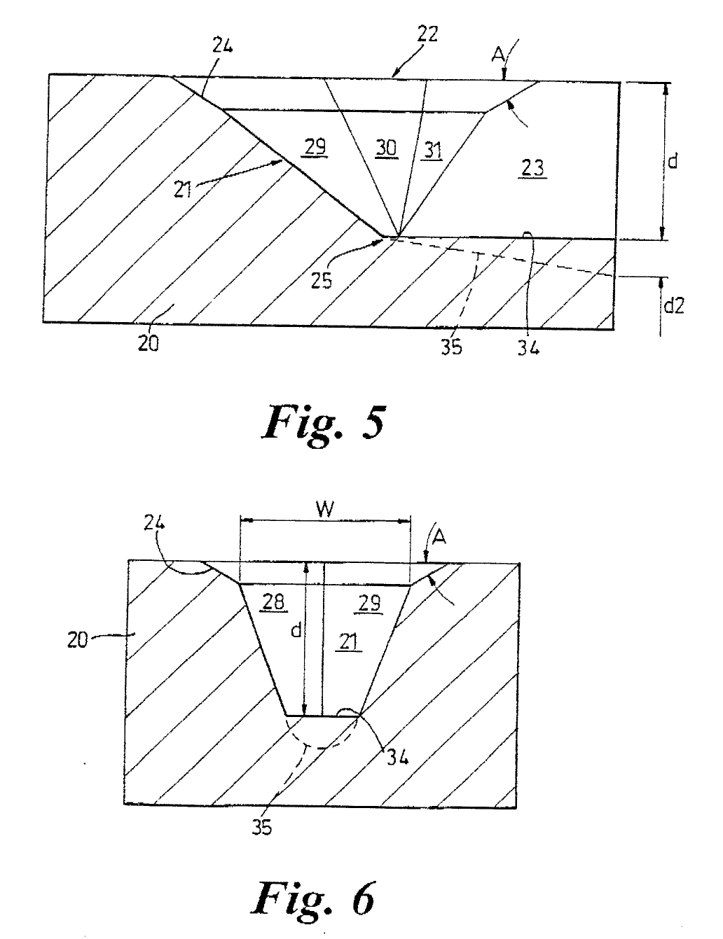

By use of the apparatus shown in the FIGS. 4 to 6, it is possible to reliably and repeatedly deflate the laryngeal mask 10 to the shape shown in FIG. 3 in which the deflated cuff 13 is flared (downwardly as shown) away from the base 15. The distal end 18 of the cuff is now substantially in line with the smooth curve of the tube 11, boss 14, and deflated cuff 13. This is a more natural shape for placement on the hard palate and therefore for insertion into the throat and larynx of a patient, as defined previously.

The apparatus shown in FIGS. 4 to 6 comprises a body 20 in the form of a rectangular block, although other shapes could be used. In one embodiment, the body 20 is formed from cast silicone rubber, although a semi-rigid polyurethane rubber or semi-rigid foam may also be suitable. The body could also be shaped from a block of foam such as polyethylene or polypropylene foam.

The body 20 is formed with a cavity 21 therein which in plan view, as shown in FIG. 4, has a mouth 22 substantially in the form of a hexagon, typically an irregular hexagon having slightly larger sides at its distal end and smaller sides adjacent slot 23 which accommodates the tube. The cavity 21 has a sufficient depth “d” of about 4.5 cms. that it can accommodate an inverted laryngeal mask 10 with he cuff or collar 13 nestled in the mouth of the cavity 21. The slot 23 extends through the body 20 from one end of the hexagonal shaped cavity 21 to provide a through passageway for the flexible tubes 11 and 16.

The fixed sized cavity 21 can only accommodate a particular range of sizes of laryngeal mask. One size of cavity may accommodate nos. 3, 4 and 5 masks, whereas a second smaller size of cavity would accommodate the smaller nos. 1, 2 and 2.5 sizes of mask. In order to accommodate a larger size of mask e.g. a no. 5 mask, a chamfered edge margin 24 extends around the periphery of the mouth of the cavity. The edge margin 24 will be about 8 to 12 mm. in width, preferably 10 mm., and the chamfer is inclined inwardly of the cavity at an angle “A” of between 20 to 45 degrees, preferably 25 to 30 degrees.

For a larger size apparatus for the group of larger sized masks, the cavity 21 is approximately 5 cms. in width “W” at its mouth, and a length “L” of about 9 cms.

The slot 23 has a flat bottom 34 that extends into the cavity 21 forming a flat base 25 in the centre of the cavity. The cavity sidewalls 26, 27, 28, 29, 30, 31, extend downwardly from the hexagonal mouth of the cavity 21 towards the base 25, so that the sidewalls 26 to 31 are inclined inwardly to provide a cavity of progressively decreasing cross-sectional area as its depth increases.

In use, an inflated inverted laryngeal mask 10 placed in the mouth of the cavity 21 with its base 15 actually in the cavity. For a size 4 and size 5 mask the inflated cuff 13 may rest on the chamfered edge margin 24. The cuff 13 is then deflated and simultaneously a light load is placed on the base 15 by the finger(s) of the operative to slowly push the mask 10 into the cavity 21 whose depth d is sufficient to accommodate this movement. The inclined sidewalls 26-31 cause the cuff to move upwardly in the cavity and take up the configuration shown in FIG. 3. The corners of the hexagon may cause the deflated cuff 13 to buckle in a predictable manner so that in the deflated condition ribs 33 of excess material locate in alignment with the corners of the hexagonal mouth.

It has been found that this buckling is not detrimental to the use of the shaped deflated mask.

The resilience of the material from which the body is made also helps force the deflated cuff 13 into the desired configuration.

A smaller size apparatus will be necessary for the group including 1, 2 and 2.5 sizes at mask. The mouth of the hexagonal cavity should have a lengths of about 5 cms (2″) and a width of about 3.75 cms (1.5″) with a chamfered edge margin of about 7-10 mm.

The body 20 could also be moulded by vacuum forming techniques so that instead of comprising a block of material with a cavity therein, it could be formed as a thin-walled hollow moulding with a cavity formed therein. If the material forming the body is relatively rigid, e.g. glass reinforced polyester resin (GAP) or polycarbonate, it may be necessary to provide the slot 23 with an inclined semi-circular bottom 35 to accommodate the boss 14 and tube 11. This may be inclined at an angle of between 10 and 15 degrees to a depth d2 of 1.5 to 2.0 cms at the outside of the block.

The body 20 may also be formed as a composite of several components which are secured together to form a thin-walled body having the required cavity shape, and supported in a surrounding surface or block.

1. Shaping apparatus for shaping the deflated profile of a laryngeal mask and which comprises a body (20) having an open mouthed cavity (21) therein, the mouth (22) of the cavity being shaped to accommodate the elliptical outline of an inflated laryngeal mask in an inverted condition, the cavity (21) having sidewalls (26, 27, 28, 29, 30, 31) which are inclined inwardly of the cavity towards the base (25) thereof, and has sufficient depth (d) to accommodate the inverted mask and air tube.

2. Apparatus as claimed in claim 1, wherein the body (20) is made from a resilient polymeric material.

3. Apparatus as claimed in claim 2, wherein the body (20) is made from one of a silicone rubber, a semi-rigid foam, a thermoplastics material.

4. Apparatus as claimed in any one of claims 1 to 3 wherein the upper sidewalls of the cavity are chamfered and said chamfered portion forms a continuous edge margin (24) around the mouth (22) of the cavity (21).

5. Apparatus as claimed in claim 4, wherein the edge margin (24) is inwardly inclined at an angle (A) of between 20-45 degrees to the horizontal.

6. Apparatus as claimed in claim 4 or claim 5, wherein the edge margin (24) has a width of between 8 mm and 13 mm (⅜ths and ½ of one inch).

7. Apparatus as claimed in any one of claims 1 to 6, wherein the mouth 922) of the cavity (21) is substantially hexagonal in plan view with one end of the cavity connected by a slot (23) open to the outside of the body (20) and which provides a passageway for the tube.

9. Apparatus as claimed in claim 8 in which the mouth (22) has the shape of an irregular hexagon having longer sides remote from the slot (23) and shorter sides adjacent the slot (23).

10. Apparatus as claimed in claim 8 and claim 9 wherein the sidewalls (26, 27, 28, 29, 30, 31) of the cavity (21) incline inwardly from the mouth (22) of the cavity to intersect with a horizontal planar surface (25) which opens into the base of the slot (23).

11. Apparatus as claimed in any one of claims 8 to 10 wherein the base (35) of the slot is inclined downwards to accommodate the boss and tube on the back face of the mask.
